(19) United States
(12) Patent Application Publication
(10) Pub. No.: US 2012/0003684 A1
(43) Pub. Date: Jan. 5, 2012

(54) Method and Means for Detecting the Activity of Osteoclasts

(75) Inventors: Peter Dieter, Dresden (DE); Anne-Helen Lutter, Leipzig (DE); Ute Hempel, Dresden (DE)

(73) Assignee: TECHNISCHE UNIVERSITAET DRESDEN, Dresden (DE)

(21) Appl. No.: 13/255,976

(22) PCT Filed: Mar. 10, 2010
(86) PCT No.: PCT/EP2010/053054

§ 371 Date: Sep. 12, 2011

(30) Foreign Application Priority Data
Mar. 13, 2009 (DE) 10 2009 013 957.5

(51) Int. Cl. C12Q 1/02

(52) U.S. Cl. 435/29

(57) ABSTRACT

The invention relates to a method and means for detecting the resorption activity of osteoclasts, in particular for use in medicine and in bioscience and pharmaceutical research. Previous methods for measuring the resorption activity of osteoclasts in vitro are difficult to quantify, are partially inflexible when used with different donor organisms and require special measuring devices for data acquisition. The method and kit according to the invention advantageously use a biomineralized matrix which contains calcium phosphate and was obtained in vitro by depositing calcium phosphate by means of osteoblasts. Osteoclasts are incubated on this matrix and the non-resorbed calcium phosphate is then quantified. The method according to the invention advantageously functions with osteoclasts of different organisms and cell types and can be easily quantified.

The invention concerns a method and means for detecting the resorption activity of osteoclasts, in particular for use in medicine as well as in bioscience research and pharmaceutical research.

Bone is a dynamic tissue which is continuously undergoing restructuring (bone resorption and bone formation). In healthy bone, bone-forming cells (osteoblasts) and bone-resorbing cells (osteoclast) are in balance; this balance, with aging, is shifting toward osteoclasts. Many bone diseases are caused by dysfunctions of the osteoclasts or disruptions of the balance and are intensively researched. In case of osteoporosis, for example, the resorption activity of osteoclasts is disturbed.

DE 10 2004 021 229 A1 discloses a method for producing bioactive osteoblast-stimulating surfaces by modification with amorphous silicon dioxide (silica) and/or silicones as well as by means of enzymatic modification with a polypeptide that comprises a silicatein-α or silicatein-β domain. On a surface modified in this way, a mineralized calcium phosphate-containing matrix is synthesized in vitro by osteoblast line SAOS-2 in the presence of β-glycerol phosphate. With the special silica-modified surface that is coated also with collagen, an improved mineralization is achieved in comparison to a matrix that is coated with collagen alone. However, in any case the mineralization that is achieved is non-uniform and does not lead to a complete coverage of the surface.

Known methods for measuring the resorption activity of osteoclasts in vitro are difficult to quantify, are partially inflexible in respect to use with different donor organisms, and require special measuring devices for data acquisition.

On the market, there are the following test kits:

1. The Osteoclast Culture Kit of Kamiya Biomedical Company (Seattle, Wash., USA) works on the basis of compact dentin disks in combination with special rat precursor cells V-1 that differentiate to osteoclasts. Dentin is similar to bone and is comprised to approximately 70% of calcium hydroxyl apatite (mainly phosphate and calcium) and to 20% of organic components (of these 90% are collagen). The remaining 10% are water. The resorption lacunae that are produced by the osteoclast activity disadvantageously can only be quantified after hematoxylin staining or by means of electron microscope. Moreover, the dentin disks are impermeable to light and therefore disadvantageously cannot be evaluated by means of fluorescence-microscopic and light-microscopic means.

2. The OAAS™ plates of OCT USA, Inc. (Buena Park, Calif., USA) are cell culture plates that are coated with synthetically produced carbonized calcium phosphate. A quantification of the osteoclast activity is possible only to a limited extent because recognition of resorption lacunae relative to the background is difficult. An evaluation is possible by means of a CCD camera but the contrast between eaten surface area and matrix is bad because the plates will stain brown and not black.

3. The BD Biocoat™ Osteologic™ Discs of BD Biosciences (Bedford, Mass., USA) is based also on plates that are coated with synthetically produced calcium phosphate which plates in this case are stained with the von Kossa method before quantification is done. The problem of this test resides in the artificial substrate that is offered to the cells and that may affect the resorption behavior of the cells. In WO 2008/153814 A1 the BD Biocoat™ Osteologic™ Discs are employed, for example.

4. Object of US 20080299604 A1 is the kit that is marketed under the name OsteoLyse™ Assay Kit of Lonza Walkersville, Inc. It is based on the quantitative determination of collagen decomposition products. Plates that are coated with europium-marked collagen are used. Measured is the collagen decomposition and the fluorescence that is caused by release of the europium. The system functions only with osteoclast precursor cells that are specifically matched to the test system; this does not allow any variability.

5. The OsteoAssay™ Human Bone Plate of Lonza Walkersville, Inc. (Walkersville, Md., USA) is based on a thin layer of adherent human bone particles that form a matrix for primary human or non-human osteoclasts. Here, also the collagen decomposition is measured in the supernatant. A direct correlation with the resorption surface area or with the number of resorption lacunae is not possible with this test.

6. The CalciFluor™ Assay of Lonza Walkersville, Inc. is based on measuring free calcium that is released during resorption. In this case, it is problematic that a special medium must be used in order to prevent that calcium, contained in normal medium, will falsify the measured results.

U.S. Pat. No. 5,849,569 discloses a substrate for culturing bone cells in vitro for determining their activity. This substrate is comprised of synthetically produced calcium phosphate that is obtained by coating by means of a sol-gel method.

WO 2007135531 A2 discloses an image processing system for evaluation of images of osteoclast activity assays.

Object of the invention is to provide a simplified and improved method, and means for performing it, for detecting and quantifying the resorption activity of osteoclast in vitro. The method should be applicable to osteoclasts that are derived from various cell lines and native cells of various species.

The present invention was arrived at based on the desire to develop a species-independent physiological osteoclast resorption assay that is easy to quantify.

The object is solved according to the invention by a method for determining the resorption activity of osteoclast with the steps:
a) incubation of osteoclasts with a bio-mineralized calcium phosphate-containing matrix that was formed by osteoblasts in vitro;
b) quantification of the un-resorbed calcium phosphate.

The term resorption activity of osteoclasts is understood as the bone-decomposing activity of the osteoclasts.

By means of the osteoblasts a native bio-mineralized bone matrix is advantageously formed in vitro on a support material, i.e., a matrix which in its composition and in its structure corresponds virtually to natural bone.

The matrix according to the invention that was obtained by biomineralization by osteoblasts in vitro differs from a coating of synthetically produced calcium phosphate in particular with respect to the following features:
1. The matrix contains (as does natural bone) approximately 10 to 30%, preferably 15 to 25%, of organic materials (e.g. collagen type I, osteonectin, osteocalcin (optionally in smaller quantities), osteopontin, proteoglycans, bone sialoprotein) and approximately 70 to 85% of inorganic substances (primarily calcium phosphate in the form of hydroxyl apatite (Ca₁₀[PO₄]₆[OH]₂)).
2. The structure of the matrix is similar to that of natural bone, i.e., it is comprised of a network of structural proteins (such as collagen type I, osteopontin) to which, by means of calcium-binding proteins (such as calciumnectin), calcium phosphate (in particular in the form of hydroxyl apatite) is bonded.

Since the matrix according to the invention in its structure and its composition matches natural bone, the invention enables advantageously testing of the resorption activity of osteoclasts under virtually physiological conditions.

The matrix according to the invention is comprised preferably (aside from residues of the cell culturing medium) solely of biologically produced components, i.e., contains no synthetically produced calcium phosphate. Also, the use of cross-linking agents or other materials for fixation of the matrix on the support material is not required.

The matrix according to the invention can advantageously be obtained on conventional cell culturing dishes (e.g., of polystyrene or polycarbonate), ceramic or metallic surfaces (such as titanium), glass or other known, preferably transparent, support materials by incubation with osteoblasts in vitro. It is advantageous that no prior coating (no pre-coating) of the surfaces is required, i.e., the cells are directly incubated on the aforementioned surfaces in the cell culturing medium. The matrix according to the invention is thus formed directly on the aforementioned surfaces. The size of the dishes (e.g., 6-well, 24-well, 48-well, 96-well, Petri dishes of all known sizes) can be selected as desired.

The term osteoblasts encompasses in this connection osteoblast cell lines including osteo sarcoma cell lines with osteoblast properties (for example, SAOS-2 cells) and native osteoblasts.

The thickness of the matrix according to the invention can advantageously be adjusted by the duration of incubation with osteoblasts. Preferably, the layer is however thin enough to allow for light-microscopic or a simple densitometric or photometric evaluation. The evaluation by means of a CCD camera, digital photo camera or a desktop scanner is possible also. The homogeneity of the matrix according to the invention, i.e., the layer contains no gaps, is advantageous. The matrix has a consistent thickness of at least 100 nm. Preferred layer thicknesses are in the range of 0.4- 0.8 μm relative to hydroxyl apatite.

The production of the matrix is realized preferably by incubation of an osteoblast cell line, preferably of SAOS-2 cells, on the support material in a medium.

The incubation of the cells is realized preferably in a standard cell culturing medium (which contains salts, amino acids, vitamins, glucose, deoxyribonucleosides and ribonucleosides and buffer substances, for example, alpha-MEM) and serum (preferably 5% to 20% serum, preferably fetal calf serum). The medium is enriched with ascorbate (preferably 100 to 500 Nmol/l) and phosphate, in particular organically bonded phosphate, preferably β glycerol phosphate (preferably 2 to 20 mmol/l). The final concentration of ascorbate is therefore preferably at least 100 μmol/l. Standard cell culturing medium (such as alpha-MEM) contains often 300 μm ascorbate/l (50 mg/l). The final concentration of ascorbate is therefore especially preferred 400 to 800 μmol/l. The preferred final concentration of β-glycerol phosphate or other organically bonded phosphate is 2 to 20 mmol/l. The concentration of calcium salts (e.g. CaCl₂×H₂O) is preferably 0.1 to 0.5 g/l, especially preferred 0.1 to 0.3 g/l.

The incubation for formation of the matrix is realized preferably for 15 to 35 days, especially preferred 20 to 30 days. The medium is preferably changed daily to every 5 days, especially preferred every 3 to 4 days, in order to ensure optimal differentiation and satisfactory density of the calcium phosphate matrix. The CO₂ concentration is preferably in the range of 4-12%, especially preferred 5-7%, and the O₂ concentration is preferably in the range of 10-30%, especially preferred 17 to 23% or 20±2% (physiological concentration).

For the application of the matrix no machine-based assistance is required. However, the production can be automated by use of pipetting robots.

A uniform coating can be monitored by determination of the calcium phosphate contents.

After completed matrix production the still contained osteoblasts (or osteocytes) are removed from the matrix. This is realized preferably by means of aqueous ammonia solution or a urea solution. Subsequently, the plates are washed with water or a suitable buffer (such as e.g. a phosphate buffer).

Onto a matrix produced in this way the osteoclasts can be applied immediately.

When the matrix is not used immediately, it can be advantageously dried and stored at room temperature. Also, storage (e.g. in PBS) at 4° C. is possible without problems as well as freezing at −20° C. to −80° C. The matrix can also be sterilized with conventional methods (e.g. UV or γ radiation). This is however not necessarily required when exclusively sterile materials are employed.

For detection of the resorption activity of the osteoclasts (also digestion activity), they are incubated on the matrix according to the invention wherein incubation can be performed in the aforementioned cell culturing medium or other known media. The test is suitable also for use on osteoclast precursor cells (or their cell lines) wherein the latter are differentiated by addition of suitable cytokines into osteoclasts on the matrix according to the invention.

The incubation is performed preferably for 3 to 20 days, depending on the cell type, respectively. During this time, the osteoclasts decompose the matrix and in particular the calcium phosphate contained therein.

The remaining calcium phosphate is subsequently quantified. By comparison with the initial value or a calibration line, the proportion of resorbed calcium phosphate is calculated, or the resorbed surface area in relation to the non-resorbed surface area,

The test can be stopped at any point in time. When doing so, either the cells can be detached or fixation of the cells on the matrix for subsequent staining may be carried out.

Preferably, stopping is done by killing and removing the osteoclasts with aqueous ammonia solution or an urea solution. The remaining matrix is subsequently washed with water or a suitable buffer.

The matrix must not be fixed for the quantification of the resorption activity. When cells on the matrix are to be observed, they can be fixed, for example, with para formaldehyde (3.7-10%), an acetone/methanol mixture, or by means of other conventional fixation methods, and subsequently stained.

For quantification of the calcium phosphate various methods can be employed.

Preferably, a silver deposition (replacement of calcium with silver) by addition of soluble silver salts in the presence of reducing agents can be employed. In this method, also referred to as von Kossa staining, a significant differentiation (already visually) between native matrix (black) and resorbed gaps (transparent) is possible. The quantification can be realized in various ways. The simplest possibility requires no special device for reading but is based on scanning the remaining matrix or support material and subsequently analyzing the light areas by means of a software, such as Image Quant or ImageJ. The advantage in this connection is that an entire well (cavity) can be analyzed and not only a partial area so that a non-uniform distribution of holes (produced by osteoclast activity) in the well have no effect on the results. The reproducibility and validity of this integrational analysis of the entire well and thus of the entire cell population is significantly improved by the homogenous coating according to the invention.

In addition to the quantification of the osteoclast activity, also a detection of cellular markers can, preferably by immunofluorescence staining or immunohistochemistry. Advantageously, the matrix according to the invention does not interfere with such a staining process and microscopic detection (e.g., with a classic fluorescence microscope or a confocal microscope). This detection is realized preferably before performing the von Kossa staining. It is advantageous that the reagents that are used for immunofluorescence staining or immunohistochemistry and partially remain on the cells (antibodies, fixation agents such as formalin, blocking agents such as bovine serum albumin, fluorescence staining agents) will not interfere with the subsequent quantification of the calcium phosphate (preferably by means of the von Kossa staining).

Alternatively, the quantification of the calcium phosphate can be done by other known detection methods for calcium (e.g., with calcon and/or calcein or arsenazo III or as hexacyanoferrate) or phosphate or also by radioactive marking of the calcium or phosphorus.

Advantageously, the method according to the invention functions equally well with osteoclasts of different organisms and cell types, in particular primary mouse osteoclast cells (primary mouse bone marrow monocytes), mouse osteoclast cell lines (for example, RAW264.7), primary rat osteoclast cells, rat osteoclast cell lines, osteoclasts that are obtained from primary human peripheral mononuclear blood cells (PBMC—peripheral blood mononuclear cells) or primary human CD14⁺cells by differentiation, primary human cells from the bone marrow (human bone marrow cells), osteoclast precursor cells from Lonza.

Already during culturing it is possible to follow by light microscope (preferred with contrast microscopy) the generation of holes. Also, different studies of the differentiation of the osteoclasts can be followed by light microscope, in particular by means of fluorescence staining.

Object of the invention is also a kit for determining the resorption activity of osteoclasts, comprising:
a) a matrix that was obtained by deposition of calcium phosphate by osteoblasts in vitro, as well as, optionally, at least one of the further components:
b) cell culturing medium, differentiation factors (e.g. RANKL),
c) fixation solution, washing buffer,
d) silver nitrate solution, reducing agent,
e) a calibration line,
f) one or several osteoclast cell lines,
g) an instruction manual,
h) program information and possibly information with respect to selection and use of a scanner.

Advantageously, the user may employ osteoclast cells of his choice. Inasmuch as osteoclast cell lines are contained in the kit, they preferably serve as comparative examples or positive controls.

The invention concerns also the use of the method, the matrix and the kit of the present invention for determining the resorption activity of osteoclasts, for use in biological or medical research (e.g., for studying osteoclast differentiation), pharmacological research (e.g., for examining and developing substances that affect the osteoclast activity and/or differentiation), and for use in medicine (e.g., for diagnosis of osteoclast activity and/or differentiation). Also, by means of the invention there is the possibility of examining co-cultures of osteoblasts and osteoclasts under physiological conditions.

### EMBODIMENTS

The invention will be explained in the following by means of figures and examples in more detail without this being limiting to the invention.

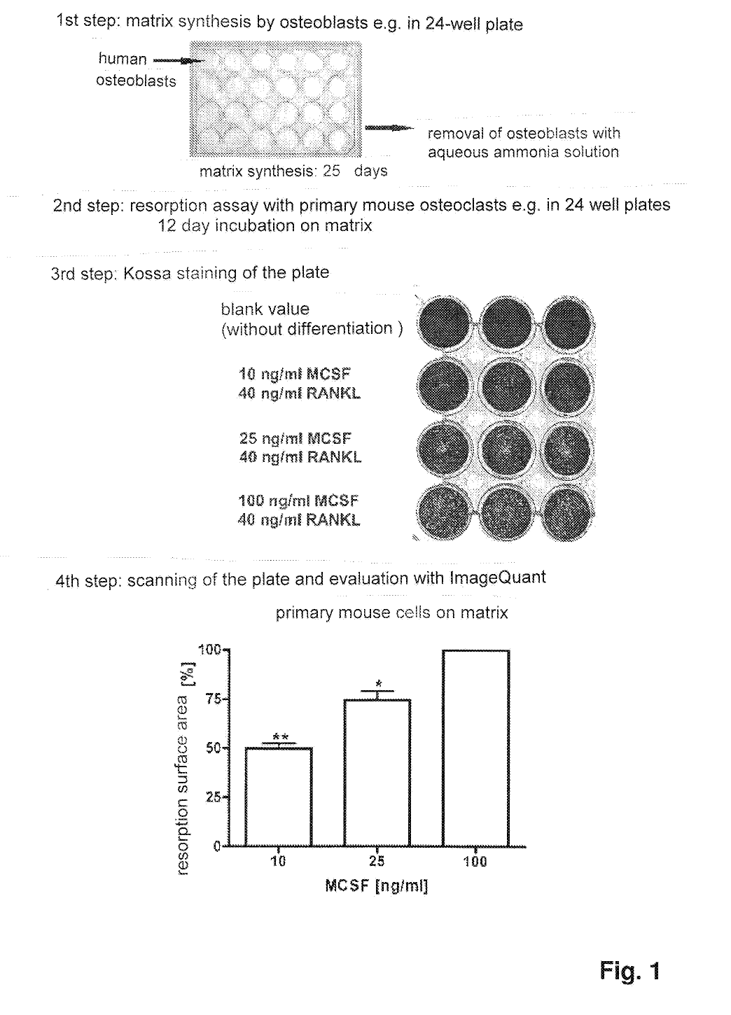

FIG. 1 shows schematically the sequence of the inventive method, including the step of matrix production. The sequence is divided into two phases. In phase I, the matrix synthesis with osteoblasts occurs; the latter, after production of the matrix (approximately 25 days), are removed from the matrix. The subsequent phase II encompasses the use of the matrix for the osteoclast resorption assay. In this connection, the method in this example was employed in order to examine the resorption activity of primary mouse osteoclast precursor cells (primary mouse bone marrow monocytes) as a function of the concentration of MCSF (macrophage colony-stimulating factor). The resorption activity of the differentiated osteoclasts (as has been demonstrated beforehand—Lees RL et al. 1999 J Bone Miner Res 14(6), 937-45) depends on the quantity of added MCSF when the differentiating factor RANKL (“receptor activator of NF-κB ligand”) (40 ng/ml) is present. The diagram of FIG. 1 shows that the examination of the MCSF dependency can be significantly assayed by means of this test.

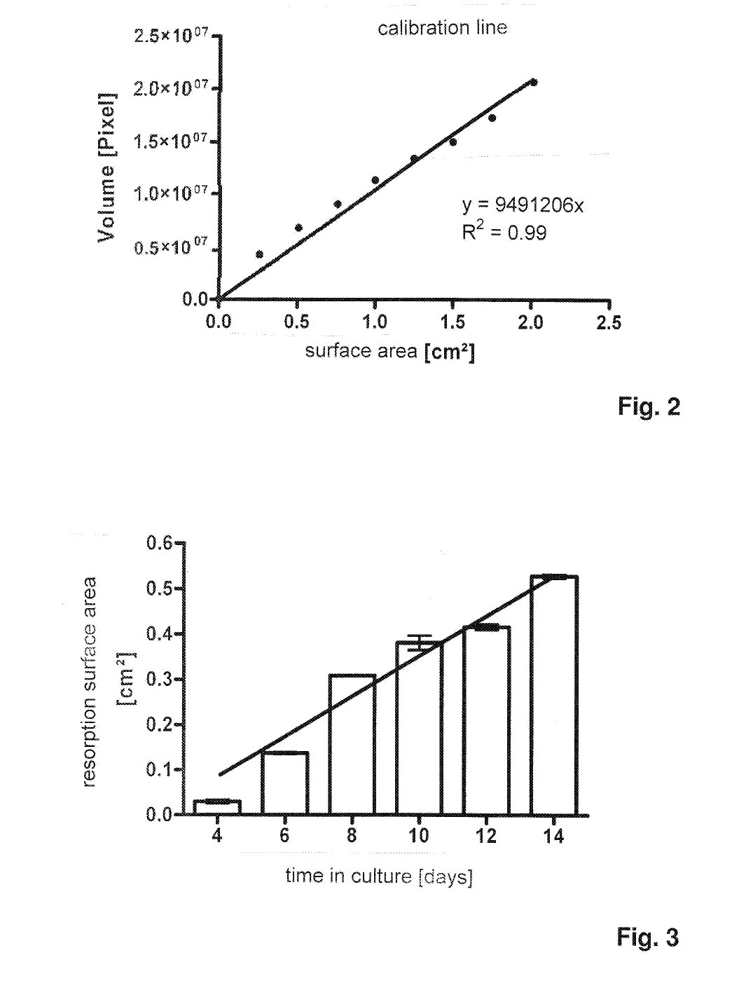

FIG. 2 shows the calibration line for calculating the resorption surface area. The calibration line is produced by partial application of the matrix.

A further example for the use of the test system is illustrated in FIG. 3. Here, the time dependency of the resorption activity of a mouse osteoclast cell line (RAW 264.7) differentiated by RANKL addition (40 ng/ml) was examined, i.e., the increase of resorption surface area per day can be simply and efficiently followed by means of this test.

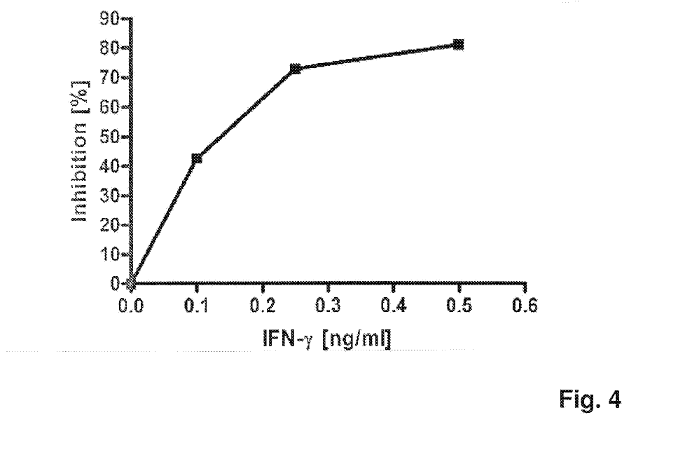

Moreover, particularly in medical research the examination of inhibiting materials or activators of osteoclast activity is especially of interest. As an example, in FIG. 4 it is shown that the osteoclast activity depends on the inhibitor interferon-y. The osteoclasts were differentiated by RANKL addition (40 ng/ml)) from a mouse osteoclast precursor cell line (RAW 264.7). In this connection, a concentration-dependent inhibition was demonstrated that has also been described already in the literature (Kamolmatyakul S, Chen W., Li YP, 2001, J. Dent. Res. 80 (1), 351-355). Accordingly, the invention can also be utilized for inhibition studies in relation to osteoclast resorption activity.

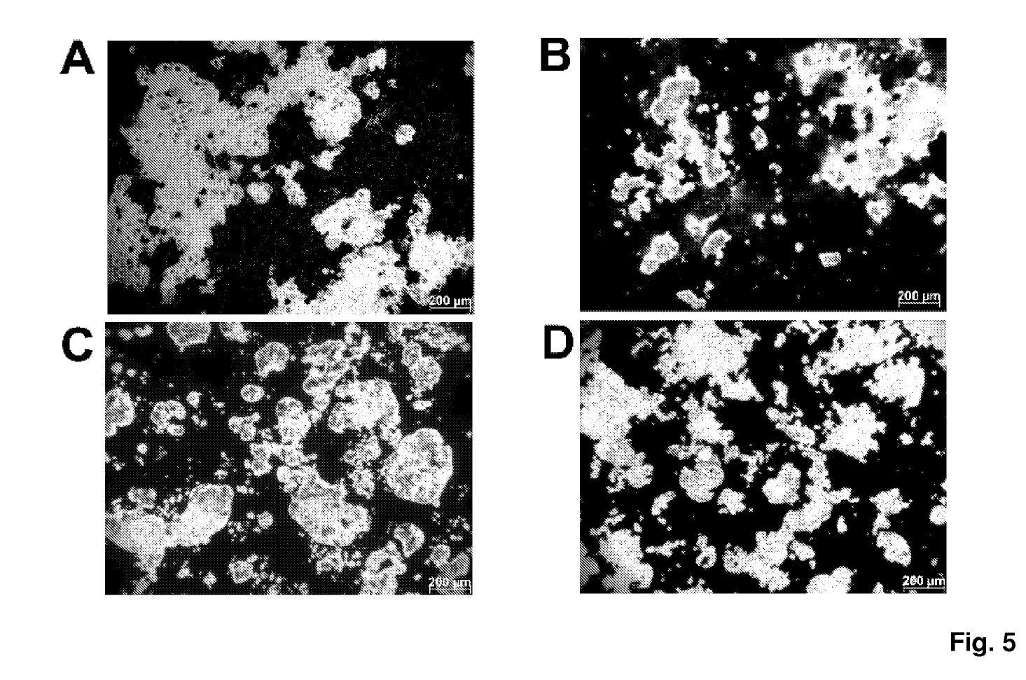

An important advantage of the developed resorption assay resides in that the cell types of different organisms can be used. FIG. 5 shows the resorbed matrix of differentiated primary human CD14⁺ PBMC (A), primary human osteoclast precursor cells of Lonza GmbH (B), RAW 264.7 cells (C), and primary mouse cells of bone marrow (D). The illustrations demonstrate that the different cell types are equally well suited to decompose the matrix.

### Example 1

### Synthesis of a Bone-Like Physiological Matrix

For the synthesis of a bone-like physiological matrix, the human osteoblast precursor cell line SAOS-2 (DSMZ ACC 243, DSMZ, Braunschweig) is used in this embodiment. The cells are seeded on cell culturing plates. The differentiation of the cells is realized by addition of ascorbate (300 μmol/l) and β-glycerophosphate (10 mmol/l) to the medium (alpha MEM, Biochrom, 10% FCS). This provides the following final concentrations: 600 μmol/l ascorbate and 10 mmol/l β-glycerophosphate. Incubation is carried out at 5% CO₂ and 20% O₂ and 37° C. The medium is exchanged every 3 to 4 days in order to ensure optimal differentiation and a sufficient density of calcium phosphate matrix. After 25 days cells that are still present are removed from the matrix with ammonia solution (20 mmol/l). The finished plates are subsequently thoroughly washed with PBS and stored at 4° C. in PBS until use.

### Example 2

### Resorption Phase by Means of Osteoclasts

In order to study the resorption behavior of active osteoclasts, different osteoclast precursor cells (see Table 1) are seeded onto the matrix plates produced in accordance with Example 1. Differentiation of the cells is realized in culture medium (alpha MEM, 20% fetal calf serum—FCS) by addition of different cytokines, depending on the cell type (see Table 1). The medium is changed every 3 days in order to ensure optimal differentiation. Independent of the cells the resorption phase is terminated after certain amounts of time. In order to enable optimal evaluation of the plates, cells that are still present are removed by ammonia solution (20 mmol/l) from the matrix. The resorbed plates are subsequently washed thoroughly with PBS and stored at 4° C. in PBS until evaluation.

TABLE 1
Osteoclasts precursor cells

| | species | medium | |
|---|---|---|---|
| primary bone marrow | murine | alpha MEM | |
| cells | | heat-deactivated FCS | 20% |
| | | penicillin/streptavidin | 10,000 U |
| | | L-alanyl-L-glutamine | 5 mmol/l |
| | | murine RANKL | 40 ng/ml |
| | | murine MCSF | 10-100 ng/ml |
| primary bone marrow | human | alpha MEM | |
| cells | | heat-deactivated FCS | 20% |
| | | penicillin/streptavidin | 10,000 U |
| | | L-alanyl-L-glutamine | 5 mmol/l |
| | | human RANKL | 10-100 ng/ml |
| | | human MCSF | 10-100 ng/ml |
| primary CD14⁺ PBMC | human | alpha MEM | |
| | | heat-deactivated FCS | 20% |
| | | penicillin/streptavidin | 10,000 U |
| | | L-alanyl-L-glutamine | 5 mmol/l |
| | | human RANKL | 10-100 ng/ml |
| | | human MCSF | 10-100 ng/ml |
| osteoclast precursor | human | Osteoclast differentiation | 10% |
| cells (Poietics | | medium (Lonza) FCS | |
| Osteoclast Precursor | | penicillin/streptavidin | 10,000 U |
| Cell System, Lonza, | | L-alanyl-L-glutamine | 5 mmol/l |
| Wuppertal, DE) | | human RANKL | 10-100 ng/ml |
| | | | (recommended by |
| | | | company: 66 ng/ml) |
| | | human MCSF | 10-100 ng/ml |
| | | | (recommended by |
| | | | company: 33 ng/ml) |
| RAW 264.7 (ATCC | murine | alpha MEM | |
| number. TIB71 ™, LGC | | heat-deactivated FCS | 20% |
| Standards GmbH, | | penicillin/streptavidin | 10,000 U |
| Wesel, DE | | L-alanyl-L-glutamine | 5 mmol/l |
| | | murine RANKL | 40 ng/ml |

### Example 3

### Evaluation of the Resorbed Plates

The resorption assay is based on the combination of two methods with each other. In this connection, the resorted plates are first stained with the von Kossa stain (von Kossa J. (1901); modified by Barkhatov I M, Roumiantsev S A, Vladimirskaya E B, Afanasyev B V (2008) Composition and functional properties of monolayer cell culture from human umbilical cord blood, Cellular Therapy and Transplantation 1 (2)). The method is based on calcium ions being replaced with silver ions which, after reduction by exposure to light, are visible as metallic silver (black). Resorted surface areas are free of calcium and therefore do not stain black while non-resorbed areas of the matrix become black. Staining was done after washing with water by incubation of the matrix with silver nitrate solution (5%) for 60 min. After repeated washing with distilled water, pyrogallol solution (1%) was used for 5 to 10 min. for development. For determining the resorption surface area the dried plates were subsequently scanned by a transmitted light scanner and evaluated e.g. by means of Image Quant. In this connection, the scanned surface area was determined and white surfaces differentiated from black surfaces. The volume of the white surface area can then be calculated by means of the calibration line (see FIG. 2) into the actual resorted surface area.

### Comparative Example

As a comparison, it was attempted to employ the OsteoLyse assay of Lonza known in the art in connection with a different osteoclast line (RAW 264.7). The kit instructions were followed in this connection.

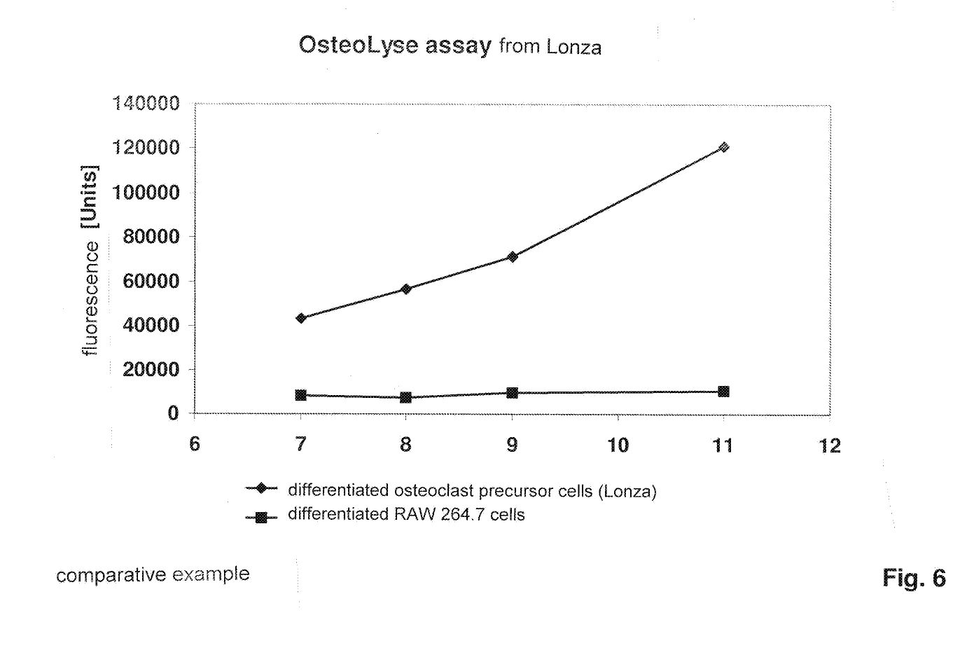

As a result FIG. 6 shows that the OsteoLyse assay of Lonza does not work with the RAW 264.7 cells while the osteoclast precursor cells of Lonza belonging to the kit exhibit a time-dependent resorption activity.

What is claimed is:

1-5. (canceled)

6. A method for determining the resorption activity of osteoclasts, the method comprising the steps of:
incubating osteoclasts with a bio-mineralized calcium phosphate-containing matrix that was obtained by osteoblasts in vitro;
quantifying un-resorbed calcium phosphate.

7. The method according to claim 6, wherein, in the step of quantifying un-resorbed calcium phosphate, staining with silver nitrate is carried out.

8. The method according to claim 7, wherein staining is carried out with von Kossa stain.

9. A method for determining the resorption activity of osteoclasts, comprising the step of providing a matrix, obtained by deposition of calcium phosphate by osteoblasts in vitro.

10. A kit for determining the resorption activity of osteoclasts, comprising:
a) a matrix that was obtained by deposition of calcium phosphate by osteoblasts in vitro, as well as at least one of the further components:
b) cell culture medium,
c) fixation solution, washing buffer,
d) silver nitrate solution, reducing agent,
e) a calibration line,
f) osteoclast cell lines,
g) an instruction manual.
